(19) United States
(12) Patent Application Publication
(10) Pub. No.: US 2007/0197780 A1
(43) Pub. Date: Aug. 23, 2007

(54) Method and apparatus for DNA purification

(76) Inventors: Jong Myeon Park, Seoul (KR); Sang Hyun Peak, Seoul (KR); Chang Eun Yoo, Seoul (KR)

Correspondence Address: CANTOR COLBURN, LLP, 55 GRIFFIN ROAD SOUTH, BLOOMFIELD, CT, 06002, US

(21) Appl. No.: 11/501,790

(22) Filed: Aug. 9, 2006

(30) Foreign Application Priority Data
Aug. 9, 2005 (KR) 10-2005-0072986

(51) Int. Cl. C07H 21/04

(52) U.S. Cl. 536/25.4

(57) ABSTRACT

A method and apparatus for DNA purification are provided which use a silicone structure having a Sio₂ layer formed on its surface. In the method, DNA is effectively purified from a fluid sample by maintaining the pH of the silicone structure's surface to be lower than the pKa of silanol to make the surface of the silicone structure charged with hydroxyl groups which results in the binding of DNA present in the fluid sample, removing the residual fluid sample which fails to bind to the surface of the silicone structure, and altering the pH of the silicone structure's surface to be higher than the pKa of silanol to separate the bound DNA from the surface of the silicone structure.

### FIELD OF THE INVENTION

The present invention relates to a method and apparatus for DNA purification. In particular, the present invention relates to a method and apparatus for DNA purification using a silicone structure having a SiO₂ layer formed on the surface thereof. The method and apparatus are capable of effectively purifying DNA from a fluid sample, such as a cell extract, by maintaining the pH of the silicone structure's surface to be lower than the pKa of silanol to make the surface of the silicone structure charged with hydroxyl groups and leading to the binding of DNA present in the fluid sample to the surface, removing the residual fluid sample which fails to bind to the surface of the silicone structure, and then changing the pH of the silicone structure's surface to be higher than the pKa of silanol to separate the bound DNA from the surface of the silicone structure.

### BACKGROUND OF THE INVENTION

Recently, as the importance of DNA analyzing techniques becomes emphasized, a technique for purifying DNA from a living body has been also regarded as an important technique and many modifications have been added thereto. A technique for efficiently purifying and concentrating DNA is very useful in a variety of applications. In particular, such a technique can be used to rapidly monitor the presence of a pathogen in blood in a very small amount, making rapid medical treatment possible. Further, analyzing beneficial information of bacterial DNA could be very useful in developing medicine or genetically engineered plants. However, since DNA contained in a living body, such as in blood, various cells or the like, is generally mixed with a variety of substances such as proteins, there is a need to perform a specific treatment step to purify only DNA therefrom. Therefore, if the overall process of DNA purification, from the isolation to the concentration of the DNA, can be conducted on a single chip considerable reductions in the time and cost for the process can be achieved.

U.S. Pat. No. 5,342,931 discloses one such technique for purifying DNA which comprises the steps of increasing hydroxyl groups on a silica surface by treating it with a strong alkaline solution, allowing DNA to bind to the treated silica surface under neutral conditions in a TE, TAE or TBE buffer, and purifying DNA therefrom with hot water or buffer.

U.S. Pat. No. 5,693,785 teaches a method for purifying DNA which comprises the steps of increasing O⁻ groups on a silica surface by treating it with a strong alkaline solution, increasing hydroxyl groups thereon through acidification (pH 4-5), allowing DNA to bind to such treated silica surface under neutral conditions in a TE, TAE or TBE buffer, and purifying DNA therefrom with hot water or buffer.

U.S. Pat. No. 5,707,799 provides a detection device for determining the presence or the content of an analyte in a test sample, wherein the surface-treated structures are arranged on a plate to fix a reactant thereto.

However, these existing methods and apparatuses for DNA purification have a problem in that they indispensably require a process of chemically treating the surface of the plate before the allowing DNA to bind to the plate.

### SUMMARY OF THE INVENTION

In order to solve the aforementioned problem of the prior art, the inventors have endeavored to develop a method for DNA purification applicable to a chip, and found that when the pH of a surface of a silicone structure having a Sio₂ layer formed on the surface thereof (in the state that no surface treatment is made) is maintained at a pH lower than the pKa of silanol, the surface becomes charged with hydroxyl groups, thereby allowing DNA present in a fluid sample such as a cell extract to bind to the surface, and when the pH of the surface is maintained at a pH higher than the pKa of silanol, DNA bound to the surface of the silicone structure is easily separated therefrom.

Accordingly, disclosed herein is a method for DNA purification. The method comprises contacting a silicone structure having a SiO₂ layer formed on a surface of the silicon structure with a fluid sample containing DNA, wherein the fluid sample has a pH lower than a pKa of silanol; removing the fluid sample from the silicone structure; and treating the silicone structure with a buffer having a higher pH than the pKa of silanol.

Also disclosed herein is an apparatus for DNA purification. The apparatus comprises a silicone structure having a SiO₂ layer formed on a surface of the silicone structure, wherein the silicone structure has a definite space, an inlet for introducing a fluid into the definite space, and an outlet for discharging the fluid from the definite space; a sample storage compartment for storing a fluid sample comprising DNA; and a buffer storage compartment for storing a buffer having a higher pH than a pKa of silanol.

The method and apparatus are capable of purifying DNA from a sample in an environment-friendly way, without using any chaotropic salt or harmful organic solvent. Additionally, the method and apparatus for DNA purification enable easy fabrication of the apparatus, with no restriction to anodic bonding necessary for coupling with other modules, such as a nucleic acid amplification part, since the silicone structure is employed as it is, without further treating the surface of the silicone structure through an additional process such as chemical coating. Furthermore, the method and apparatus for DNA purification can be implemented as a process-on-a-chip or a lab-on-a-chip by employing a microfluidics technique.

### BRIEF DESCRIPTION OF DRAWINGS

FIG. 1 is a schematic diagram showing the presence of DNA binding according to the change in the surface condition of a silicone structure having a Sio₂ layer formed on its surface according to the pKa of silanol in a method for DNA purification using the silicone structure in accordance with the present invention;

FIG. 2 is an enlarged photograph showing an example of a pillar type which is formed on the surface of the silicone structure having a SiO₂ layer formed on its surface used in a method and apparatus for DNA purification in accordance with the present invention;

FIG. 3 is a schematic diagram showing functional elements of an apparatus for DNA purification using a silicone structure having a SiO₂ layer formed on its surface in accordance with the present invention;

FIG. 4 is a graph showing the measured result of the picogreen content of two eluates obtained in each step of the method for DNA purification using a silicone structure having a SiO₂ layer formed on its surface and using picogreen-labelled genomic DNA (gDNA) (2.5 ng/μl) , wherein the eluates are obtained under the pH condition lower than pKa of silanol and under the pH condition higher than pKa of silanol, respectively;

FIG. 5 is a graph showing the results of determining the protein content of the final eluate obtained in purifying DNA from a cell lysate of E. coli using an apparatus and method for DNA purification disclosed herein and the eluates obtained by using a CST (Charge Switch Technology) DNA purification kit of DRI (DNA Research Innovations, Inc., UK) and a DNA purification kit of Quiagen; and

FIG. 6 is a graph comparing the PCR result of the final eluate obtained in purifying DNA from a cell lysate of E. coli using an apparatus and method for DNA purification disclosed herein with those of eluates obtained by using commercial DNA purification kits manufactured by DRI (DNA Research Innovations, Inc., UK) and Quiagen.

### DETAILED DESCRIPTION OF THE INVENTION

The invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

To accomplish the above objects of the present invention, there is provided a method for DNA purification using a silicone structure having a SiO₂ layer formed on its surface. The method comprises the steps of: contacting the silicone structure having a SiO₂ layer formed on a surface with a fluid sample containing DNA, wherein the fluid sample has a pH lower than the pKa of silanol; removing the unbound components of the fluid sample from the surface of the silicone structure; and treating the surface of the silicone structure with a buffer having a higher pH than the pKa of silanol after removing the unbound components of the fluid sample.

The silicone structure used in the method and apparatus for DNA purification has a SiO₂ layer formed on a surface thereof and has no limitation on its configuration. However, it is preferable to fabricate the silicone structure to have a surface area as large as possible for surface contact with a fluid sample since a larger surface area contacting a fluid sample containing DNA favors binding between the DNA and the surface of the silicone structure.

When the surface of the silicone structure having a SiO₂ layer formed on it contacts a fluid sample containing DNA at a pH condition lower than the pKa of silanol, the surface of the silicone structure becomes charged with hydroxyl groups, thereby leading to the binding of DNA thereto. Preferably, the pH of the fluid sample ranges from pH 3 to 6.5. If the pH is lower than pH 3, DNA becomes damaged, and if the pH is higher than pH 6.5, the surface of the silicone structure having a SiO₂ layer formed on irs surface is not charged with hydroxyl groups. Buffers having a pH ranging from 3 to 6.5 include, but are not limited to, formate, citrate, succinate, acetate and the like.

Further, in order to maintain the pH of the fluid sample to be lower than the pKa of silanol, it is preferable to regulate the sample's pH in advance by using one of the aforementioned buffers in the course of preparing the fluid sample.

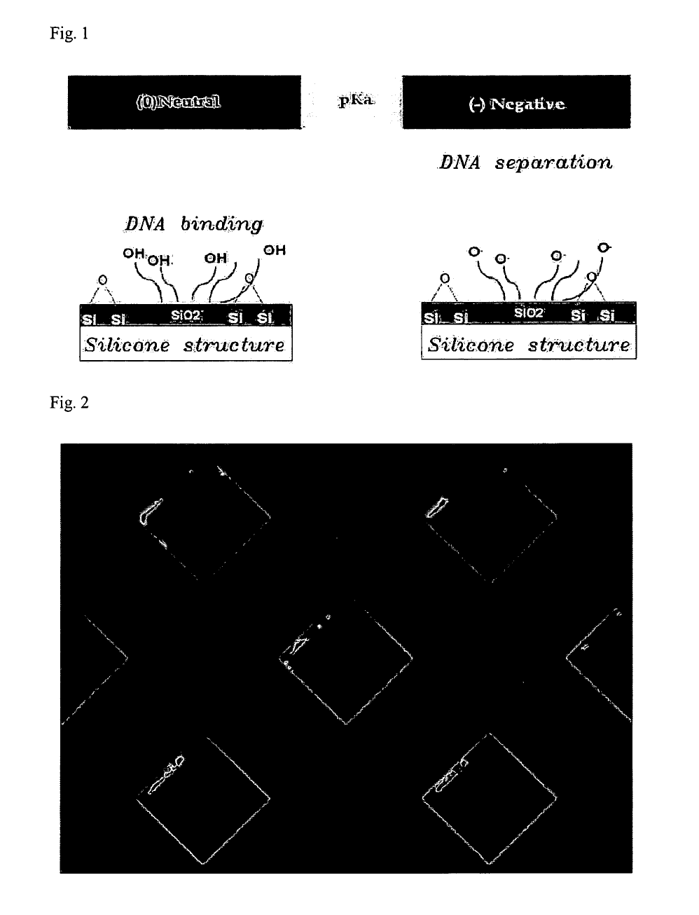

Accordingly, the step of contacting the silicone structure having a SiO₂ layer formed on a surface thereof with a fluid sample containing DNA and having a pH lower than the pKa of silanol makes the surface of the silicone structure charged with hydroxyl groups. Allowing the contact between the surface and the fluid sample to continue for a certain period of time leads to the binding of DNA in the fluid sample to the surface of the silicone structure, as described in FIG. 1. The surface of the silicone structure having the SiO₂ layer is charged with hydroxyl groups (acidic condition), rather than with positively charged groups, in order to modulate the electrostatic interaction between DNA and the surface of the silicone structure and weaken the binding force between the silicone structure and DNA, thereby weakening the force necessary to separate the DNA from the surface.

Unbound components of the fluid sample are then removed from surface of the silicone structure. In order to purify only DNA bound to the surface of the silicone structure, it is preferable after removing the unbound components of the fluid sample from contact with the surface to wash the surface. It is desirable to wash the surface of the silicone structure with a buffer having a pH lower than the pKa of silanol, i.e., pH 3 to 6.5.

Subsequently, DNA purification is completed by separating DNA bound to the surface of the silicone structure from the silicon structure's surface. To separate DNA bound to the surface of the silicone structure, the suface of the silicone structure is treated with a pH condition higher than the pKa of silanol. Specifically, the pH higher than pKa of silanol ranges from pH 8 to 10; preferable buffers include, but are not limited to, phosphate, borate, carbonate, etc.

The treating step is carried out by wetting the surface of the silicone structure with a buffer having a pH of 8 to 10 in order to charge the surface of the silicone structure with SiO⁻, thereby causing separation of previously bound DNA from the surface, as described in FIG. 1. As a result, after the treating step, the buffer obtained or eluted from the surface contains only DNA, and the DNA purification is completed.

On occasion, it may be possible that the method of the present invention comprises a pre-treating step in advance of other steps in order to facilitate the binding of DNA to the silicone structure having a Sio₂ layer formed on a surface thereof. Specifically, the pre-treating step removes air on the surface of the silicone structure, and at the same time, makes the surface of the silicone structure charged with hydroxyl groups. This is achieved by pre-treating the surface with a buffer having a pH lower than the pKa of silanol, i.e., pH 3 to 6.5, prior to contacting the silicone structure with the fluid sample containing DNA. When the pre-treating step is carried out in advance, it is possible to reduce the time of contact between the silicone structure and the fluid sample containing DNA.

In an embodiment of the method, the silicone structure having a Sio₂ layer formed on the surface thereof is fabricated in a pillar type. The pillar type is fabricated in the form of a column structure which is formed by erecting the surface itself of the silicone structure towards the upper direction from the flat. To enlarge the potential contact surface area, the configuration and interval of the column structure can be regulated as required, and a pillar type as depicted in FIG. 2 may be proposed as a preferred embodiment. To fabricate the surface of the silicone structure in a pillar type can be accomplished by utilizing a known process such as an etching.

Further, the present invention provides an apparatus for DNA purification which comprises: a silicone structure having a SiO₂ layer formed on a surface thereof, wherein the silicone structure comprises a definite space, an inlet capable of introducing a fluid into the definite space, and an outlet capable of discharging the fluid from the definite space; a sample storage compartment for storing a fluid sample comprising DNA; and a buffer storage compartment for storing a buffer having a higher pH than the pKa of silanol.

In an embodiment of the apparatus, the overall shape of the silicone structure having a SiO₂ layer formed on a surface thereof is fabricated as a chamber type. In particular, the definite space of the silicone structure is fabricated in a state such that the surface of each of the sides forming the chamber can be contacted with a fluid introduced into the chamber through the inlet. Preferably, the surface of each of the sides forming the chamber is fabricated in a pillar type in order to increase the contact area between the fluid and the surface of the silicone structure to enhance DNA binding and purification capacity.

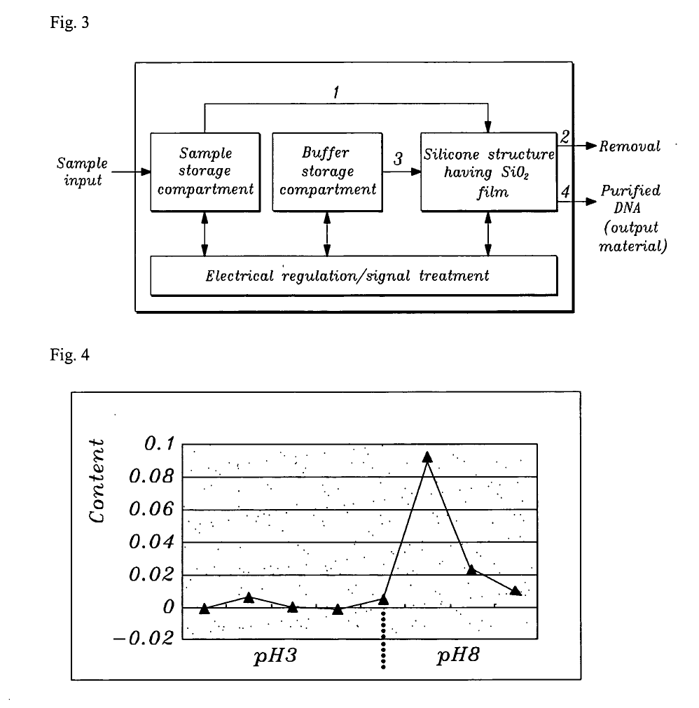

As can be seen in FIG. 3, the apparatus for DNA purification comprises three functional elements: a sample storage compartment; a buffer storage compartment and a silicone structure having a SiO₂ layer formed on a surface thereof. The apparatus can further comprise microfluidic units connecting between the functional elements although this is not represented in FIG. 3.

Further, in other embodiments, the apparatus can further comprise as functional elements a pre-treatment buffer storage compartment for storing a buffer having a lower pH than the pKa of silanol to pre-treat the surface of the silicone structure or an eluate storage compartment for storing an eluate, for example an eluate having a higher pH than the pKa of silanol, which is discharged from the silicone structure through the outlet.

Since the present invention mainly pertains to samples of biomaterials, the sample storage compartment is embodied in such a way that a fluid sample containing DNA to be purified can be introduced into the sample storage compartment from outside the apparatus. Similarly, the buffer storage compartment and the pre-treatment buffer storage compartment are also fabricated in such a manner that a buffer can be introduced to the compartment from outside the apparatus.

Further, in some embodiments, the eluate storage compartment can be fabricated in a manner to permit connection to a DNA amplification part, such as a PCR chip, to provide DNA to be amplified therein.

In an embodiment, the microfluidic units of the apparatus can comprise: a connection part connecting each of the sample storage compartment, buffer storage compartment, the pre-treatment buffer storage compartment and the eluate storage compartment to the inlet or the outlet of the silicone structure; control parts that are installed between the silicone structure and each of the sample storage compartment, the buffer storage compartment, the pre-treatment buffer storage compartment and the eluate storage compartment to control the opening/closing of each in response to a specific signal; and a driving part which provides a driving force to transport a fluid from any of the sample storage compartment, the buffer storage compartment, the pre-treatment buffer storage compartment and the eluate storage compartment to the silicone structure or to transport an eluate in a fluid state from the silicone structure to the eluate storage compartment.

The microfluidic units of the present invention employ technical elements well-known in the art. In particular, it is preferable that the connection part is a micro-channel having a size greater than a diameter through which DNA included in the fluid sample can be passed, each of the control parts is a flap valve which controls the flow of a fluid by regulating the opening/closing of the flap by using a driving force as a kind of active valve, and the driving part is a micropump.

Hereinafter, the role of each functional element in the apparatus for DNA purification will be described in sequence with reference to FIG. 3.

First, a sample containing DNA is introduced into the sample storage compartment and stored therein (although not described in FIG. 3, a buffer can also be introduced into the buffer storage compartment for its storage). There is no limitation on the nature of the introduced sample so long as it contains DNA to be purified, however in some embodiments, the sample is a cell extract. Further, the sample introduced into the sample storage compartment is in a fluid state and has a pH lower than the pKa of silanol; specifically, the pH ranges from pH 3 to 6.5.

The fluid sample kept in the sample storage compartment is introduced to the definite space of the silicone structure through the connection part which is coupled with the inlet of the silicone structure. The fluid sample is introduced in an amount sufficient to fill only the definite space of the silicone structure. When the fluid sample contacts the surfaces forming the definite space of the silicone structure, the surfaces become charged with hydroxyl groups. Then, DNA in the fluid sample binds to the surfaces, a process taking from around a minute to several minutes depending on the surface area of the definite space. Then, the fluid sample is removed from the definite space through the outlet of the silicone structure. In an embodiment, after charging the surfaces, the fluid sample is fed into the definite space at a regular flow rate to induce the binding of DNA to the charged surfaces.

Next, a buffer having a pH higher than the pKa of silanol stored in the buffer storage compartment is introduced to the definite space of the silicone structure through the connection part coupled with the inlet of the silicone structure in an amount sufficient to fill only the definite space of the silicone structure. The buffer contacts the surfaces forming the definite space of the silicone structure, thereby making the surfaces charged with SiO⁻. After allowing the buffer to stand for a while in the definite space, DNA is released from the surfaces of the definite space, allowing purified DNA to be acquired. The release of DNA takes from around a minute to several minutes depending on the surface area of the definite space. In some embodiments, in order to release DNA bound to the surface of the silicone structure, the buffer is introduced into the definite space at a regular flow rate over a certain time.

In other embodiments of the apparatus for DNA purification, the apparatus can further comprise a pre-treatment buffer storage compartment or an eluate storage compartment (not shown in FIG. 3).

When using an embodiment of the apparatus comprising a pre-treatment buffer storage compartment, the method can comprise two further step sin addition to the aforementioned steps. The method can comprise introducing a buffer into the pre-treatment buffer storage compartment. The method can further comprise introducing the pre-treatment buffer having a pH condition lower than the pKa of silanol to the definite space of the silicone structure in an amount sufficient to fill the definite space. The introduction can be through the connection part for the pre-treatment buffer storage compartment coupled with the inlet of the silicone structure. When the surfaces forming the definite space of the silicone structure contact the pre-treatment buffer, air present in the definite space is completely removed and the surfaces forming the definite space are charged with hydroxyl groups. Further, the buffer can be introduced at a regular flow rate to charge all the surfaces for a time ranging from several seconds to several minutes, depending on the surface area of the definite space.

Subsequently, the same procedure as described above is carried out to purify DNA from the fluid sample. However, in order to remove unbound components of the fluid sample from the surfaces forming the definite space of the silicone structure more completely, the pre-treatment buffer having a lower pH than the pKa of silanol can be used to wash the surfaces of the definite space. To wash the definite space after removal of unbound components of the fluid sample, the pre-treatment buffer can be introduced in an amount sufficient to fill the definite space of the silicone structure through the connection part coupled with the inlet of the silicone structure. The pre-treatment buffer can then be discharged through the outlet. Such a washing step is an alternative to introducing the buffer having a higher pH than the pKa of silanol into the definite space of the silicone structure immediately after removing the unbound components of the fluid sample from the definite space.

The buffer having a higher pH than the pKa of silanol discharged through the outlet in the final step contains only DNA in a purified state. This DNA-containing eluate can be kept in the eluate storage compartment and used as a template substance for other experiments, such as DNA amplification. Therefore, in an embodiment, the apparatus can be configured to connect the eluate storage compartment to a DNA amplification part.

The apparatus for DNA purification disclosed herein can be implemented as a process-on-a-chip or a lab-on-a-chip by implementing each functional element of the apparatus using microfluidics techniques and MEMS devices well-known in the art.

The method and apparatus for DNA purification using a silicone structure having a Sio₂ layer formed on a surface thereof have the following benefits.

The method and apparatus for DNA purification can purify DNA from a sample by using variation in the pH conditions to bind the DNA in the sample to the surface of the silicone structure and then to displace the bound DNA bound from the surface.

The method and apparatus for DNA purification can purify DNA from a sample in an environment-friendly way, without using any chaotropic salt or harmful organic solvent.

The method and apparatus for DNA purification employ the surface of the silicone structure as it is, without further processing it with an additional step such as chemical coating. Therefore, fabrication of the apparatus is easy and there is no limitation to anodic bonding process which is required for the binding to other modules such as a nucleic acid amplification part.

The method and apparatus for DNA purification can be implemented as a process-on-a-chip or a lab-on-a-chip by using microfluidics techniques, which curtails expenses. Furthermore, it is possible to manufacture a DNA amplification part, a detection part and a DNA analyzer, as well as the apparatus for DNA purification of the present invention, on a semiconductor plate as a single body.

Hereinafter, the present invention will be described in detail with reference to the following examples, which are provided as its illustrations merely but not intended to limit the scope of the present invention.

### EXAMPLE 1

A fluid sample at pH 3 containing gDNA (2.5 ng/μl) labeled with picogreen, wherein the gDNA was previously purified from a biological source and subsequently labeled with picogreen was used and was subjected to each purification step of the method for DNA purification using an apparatus comprising a silicone structure having a SiO₂ layer formed on a surface thereof. Eluates having a pH lower or higher than the pKa of silanol were obtained. For this example, the pH of the buffer kept in the buffer storage compartment of the apparatus was pH 8.

The content of picogreen in each eluate of a given pH was measured and is shown in FIG. 4.

As shown in FIG. 4, the content of picogreen measured in the eluate having a pH lower than the pKa of silanol, i.e., pH 3, was close to zero, which shows that when the pH of the surface of the silicone structure was lower than the pKa of silanol, DNA was bound to the silicon structure's surface and did not elute.

In contrast, the content of picogreen measured in the eluate having a pH higher than the pKa of silanol, i.e., pH 8, was nearly 0. 1, showing that when the pH of the surface of the silicone structure is higher than the pKa of silanol, DNA was separated from the silicon structure's surface (i.e., no longer bound to the surface) and could be eluted from the silicon structure.

### TEST EXAMPLE 1

Measurement of PCR Efficiency of DNA According to pH Condition

In order to examine whether PCR efficiency of purified DNA varies with the pH of the DNA sample, gDAN sample (2.5 ng/μl) at pH 3, GDNA sample (2.5 ng/μl) at pH 8, wherein the gDNA was previously purified from a biological source and subsequently labeled with picogreen were compared in LIGHTCYCLER PCR. Additionally, LIGHTCYCLER PCR was run on a sample lacking DNA and on a sample of the DNA-containing pH 8 eluate obtained in Example 1. The results are shown in Table 1 below.

TABLE 1
LIGHTCYCLER PCR execution results

| Conditions | PCR results (CP) |
|---|---|
| gDNA sample (2.5 ng/μl) at pH 3 | 14.70 |
| gDNA sample (2.5 ng/μl) at pH 8 | 14.80 |
| Eluate at pH 8 | 15.06 |
| Completely free of DNA | 25.68 |

As can be seen from lines 1 and 2 of Table 1 above, there is little difference in PCR efficiencies of purified DNAs with pH of the DNA added to the reaction. Further, the PCR result of DNA purified using the method and apparatus for DNA purification of the present invention is little different from that of the gDNA sample (2.5 ng/μl) at pH 8. From these results, it can be deduced that DNA purification from a sample using the method and apparatus for DNA purification using a silicone structure having a SiO₂ layer formed on its surface is effective.

### EXAMPLE 2

E. coli cells were prepared as a sample by subjecting the cells to boiling lysis four times. Boiling lysis comprises heating the cells at 95° C. for 1 min and then cooling at 30° C. for 30 sec. The cell lysate prepared by this method had a cell concentration of 1 to 2×10⁻⁹/ml. Thereafter, the cell lysate thus prepared was subjected to each step of the method for DNA purification using the apparatus comprising the functional elements described in FIG. 3 to yield a DNA-containing eluate.

### COMPARATIVE EXAMPLE 1

Comparative purified substance 1 was obtained by taking a cell lysate as described in Example 2 and subjecting the sample to purification using a DNA purification kit manufactured by Quiagen. The manufacturer's protocol was followed.

### COMPARATIVE EXAMPLE 2

Comparative purified substance 2 was obtained by taking a cell lysate as described in Example 2 and subjecting the sample to purification using a CST (Charge SwitchTechnology) DNA purification kit manufactured by DRI (DNA Research Innovations, Inc., UK). The manufacturer's protocol was followed.

### TEST EXAMPLE 2

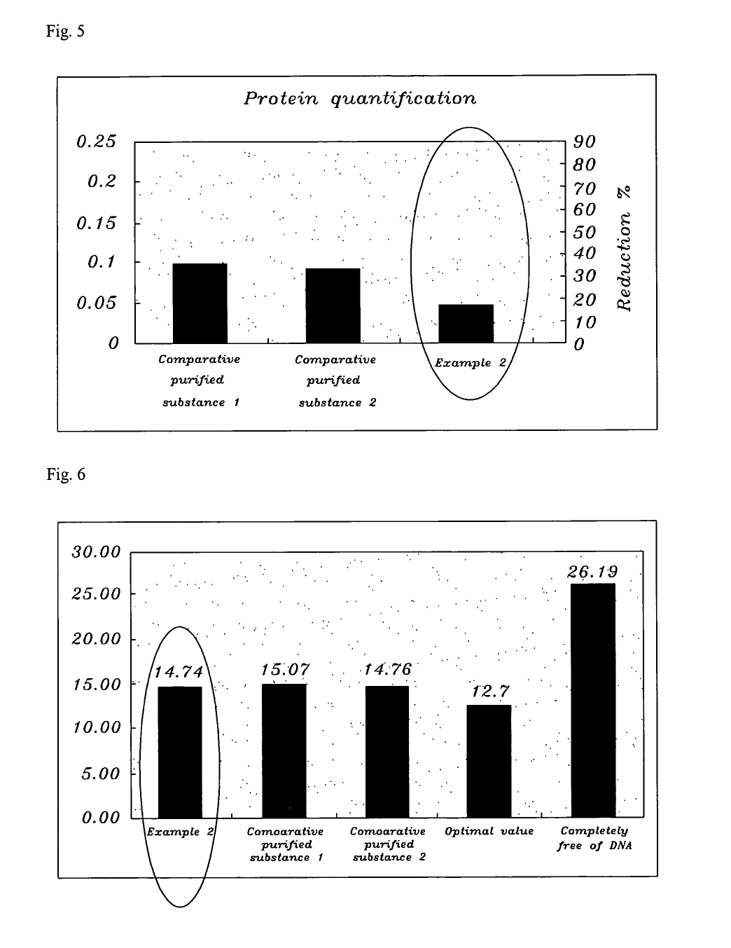

The amount of protein removed from the sample in the course of DNA purification was measured by comparing the protein content of the original lysate to that of the eluate obtained in Example 2 and of comparative purified substances 1 and 2 obtained in Comparative Examples 1 and 2. The results are described in Table 2 below and presented in FIG. 5. Each of the above measurements was conducted three times. The results represented are the average of the three measurements.

TABLE 2
Results of protein quantification

| Conditions | Protein reduction rate (%) |
|---|---|
| Eluate obtained in Example 2 | 79 |
| Comparative purified substance 1 | 57 |
| Comparative purified substance 2 | 60 |

From the results shown in Table 2 above and in FIG. 5, it can be seen that the protein content measured in the eluate containing the purified DNA obtained using the method and apparatus for DNA purification disclosed herein is about 20% less than those of the purified substances obtained using the commercial DNA purification kits of Quiagen and DRI. This larger reduction in protein present in the eluate obtained in Example 2 shows that the method and apparatus for DNA purification of the present invention permits a higher level of purification of the DNA than the two commercial methods and kits used.

### COMPARATIVE EXAMPLE 3

The eluate obtained in Example 2, and the comparative purified substances 1 and 2 obtained in Comparative Examples 1 and 2 were subjected to LIGHTCYCLER PCR, respectively. The results are shown in Table 3 below and in FIG. 6. The results presented are the average of three amplification reactions per sample.

TABLE 3
LIGHTCYCLER PCR execution results

| Conditions | PCR results (CP) |
|---|---|
| Eluate obtained in Example 2 | 14.74 |
| Comparative purified substance 1 | 15.07 |
| Comparative purified substance 2 | 14.76 |
| Optimal value | 12.70 |
| Completely free of DNA | 26.19 |

As can be seen from Table 3 above and FIG. 6, the PCR result of the eluate containing the purified DNA obtained using the method and apparatus for DNA purification of the present invention is closer to the optimal value (the CP for PCR when conducted most efficiently) than those of the purified substances obtained using the commercial DNA purification kits of Quiagen and DRI, showing superior purification efficiency of the method and apparatus for DNA purification of the present invention.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for DNA purification, which comprises:
contacting a silicone structure having a SiO₂ layer formed on a surface thereof with a fluid sample containing DNA, wherein the fluid sample has a pH lower than a pKa of silanol;
removing the fluid sample from the silicone structure; and
treating the silicone structure with a buffer having a higher pH than the pKa of silanol.

2. The method according to claim 1, which further comprises
pre-treating the surface of the silicone structure with a pH condition lower than the pKa of silanol.

3. The method according to claim 1, wherein the pH of the fluid sample ranges from 3 to 6.5.

4. The method according to claim 1, wherein the pH of the buffer ranges from 8 to 10.

5. The method according to claim 4, wherein the buffer is selected from the group consisting of phosphate, borate and carbonate.

6. The method according to claim 1, wherein the surface of the silicone structure is fabricated in a pillar type.

7. An apparatus for DNA purifications, which comprises:
a silicone structure having a SiO₂ layer formed on a surface of the silicone structure,
wherein the silicone structure comprises a definite space, an inlet for introducing a fluid into the definite space, and an outlet for discharging the fluid from the definite space;
a sample storage compartment for storing a fluid sample comprising DNA; and
a buffer storage compartment for storing a buffer having a higher pH than a pKa of silanol .

8. The apparatus according to claim 7, wherein an inner surface of the definite space of the silicone structure is fabricated in a pillar type.

9. The apparatus according to claim 7, which further comprises
a pre-treatment buffer storage compartment for storing a buffer to pre-treat the silicon structure.

10. The apparatus according to claim 7, which further comprises
an eluate storage compartment for storing an eluate which is discharged from the silicone structure through the outlet.

11. The apparatus according to claim 10, wherein the eluate storage compartment is connected to a DNA amplification part.

12. A lab-on-a-chip comprising the apparatus for DNA purification of claim 7.
